(19) United States
(12) Patent Application Publication
(10) Pub. No.: US 2014/0243954 A1
(43) Pub. Date: Aug. 28, 2014

(54) TRANSCATHETER MITRAL VALVE PROSTHESIS

(71) Applicant: Donald Shannon, Trabuco Canyon, CA (US)

(72) Inventor: Donald Shannon, Trabuco Canyon, CA (US)

(21) Appl. No.: 14/192,053

(22) Filed: Feb. 27, 2014

Related U.S. Application Data
(60) Provisional application No. 61/770,030, filed on Feb. 27, 2013.

(51) Int. Cl. A61F 2/24

(52) U.S. Cl. CPC A61F 2/24
USPC 623/2.11

(57) ABSTRACT

An intraluminal device replaces the mitral valve in the human heart without tissue insult or injury via catheter-based deployment and securement. By controlling radial force, a one-piece device in a substantially hyperboloid configuration is emplaced using a least one of a biological adhesive and a porous layer encapsulating the device, for example, ePTFE, to manage the potential for migration. Systems for emplacement are likewise disclosed.

### BACKGROUND OF THE DISCLOSURE

The present disclosure relates to devices, systems and methods for addressing, mitigating and extenuating mitral valve issues and failures. In particular, the present disclosure provides users with a minimally invasive heart valve.

### FIELD OF THE DISCLOSURES

Apparatus, systems, devices and tools to replace the mitral valve in the human heart. Using intraluminal and minimally invasive techniques devices according to the instant teachings are emplaced within beating hearts.

### SUMMARY OF THE DISCLOSURE

Briefly stated, an intraluminal device replaces the mitral valve in the human heart without tissue insult or injury via catheter-based deployment and securement. By controlling radial force a one-piece device in a substantially hyperboloid configuration is emplaced using a least one of a biological adhesive and a porous layer encapsulating the device, for example, ePTFE, to manage the potential for migration. Systems for emplacement are likewise disclosed.

According to embodiments, there is provided a device for replacing the mitral valve, comprising, in combination, a modified hyperboloid self-expanding stent-like assembly having a top and a bottom end, operatively linked to at least one of a biological adhesive and a porous coating layer, a catheter-based delivery system, and, wherein the bottom or pressure side of the assembly is modified to support integration with chordea tendineae for anchorage enhancement.

According to embodiments, there is wherein the system is adjustable.

According to embodiments, there is provided a kit, comprising at least a said device for replacing the mitral valve, a quantum of said biological adhesive, a self-expanding stent like framework which is sized to correctly fit the orifice where the host mitral valve resides, and tools for emplacement of said items.

### BRIEF DESCRIPTION OF THE DRAWINGS

FIG. 1 is a schematic view of an assembly according to the present invention which comprises a self-expanding stent-like framework sized correctly to fit the orifice wherein the host mitral valve resides according to the instant teachings.

### DETAILED DESCRIPTION OF THE DISCLOSURE

The present inventor has discovered that the ability to deploy from a catheter and secure in place without applying damaging radial force to the surrounding tissue is a key to replacing challenged mitral valves. Radial force could restrict blood flow around the valve area causing a heart attack or ischemia. The device must seal to the host tissue and not migrate due to increased physical activities.

An intraluminal device meant to replace the mitral valve in the human heart. The concept of this device is to physically secure a replacement mitral valve in the beating heart replacing the host's present mitral valve. The device can be either a one or two piece assembly. The inventive principle is the ability to deploy from a catheter and secure in place without applying damaging radial force to the surrounding tissue. Radial force could restrict blood flow around the valve area causing a heart attack or ischemia. The device must seal to the host tissue and not migrate due to increased physical activities.

The optimal device would be a one piece assembly which deploys and secures mechanically in the orifice where the present mitral valve resides. There two obstacles to overcome.

First, radial force must be controlled. This will be accomplished by using a self-expanding stent like framework which is sized to correctly fit the orifice where the host mitral valve resides. This can be from just a few millimeters of over size to a few millimeters undersize. This framework must be anchored to the tissue which is weak and diseased. Without anchoring, the stent like framework will dislodge. The anchoring cannot be done using radial force. The anchoring must be done mechanically by designing the stent like framework to appear similar to look like two coffee saucers secured bottom to bottom. Geometrically the configuration would be called a modified hyperboloid where the bottom or pressure side of the assembly may have to be modified further to account for the chordae in the heart that support the leaflet tissue and anchoring the host mitral valve. The placement to the stent like framework would allow a solid diameter framework for a new mitral valve to function. The new valve could be placed as a secondary step or placed as one continuous deployment.

Second, migration must be addressed. Since the placement of the stent like framework is a “loose fit” to the surrounding tissue so as to not cause heart failure, the stent like framework must be secured over the long term life to the patient. A biological adhesive material may hold for the short term. It may also cause interactions that are damaging to some patients. The long term fix is a material ‘like ePTFE’ bonded or encapsulating the stent like framework which encourages tissue attachment/tissue ingrowth to the stent like framework and secures it in place indefinitely.

Once the healing process is complete, normal activities can be freely performed.

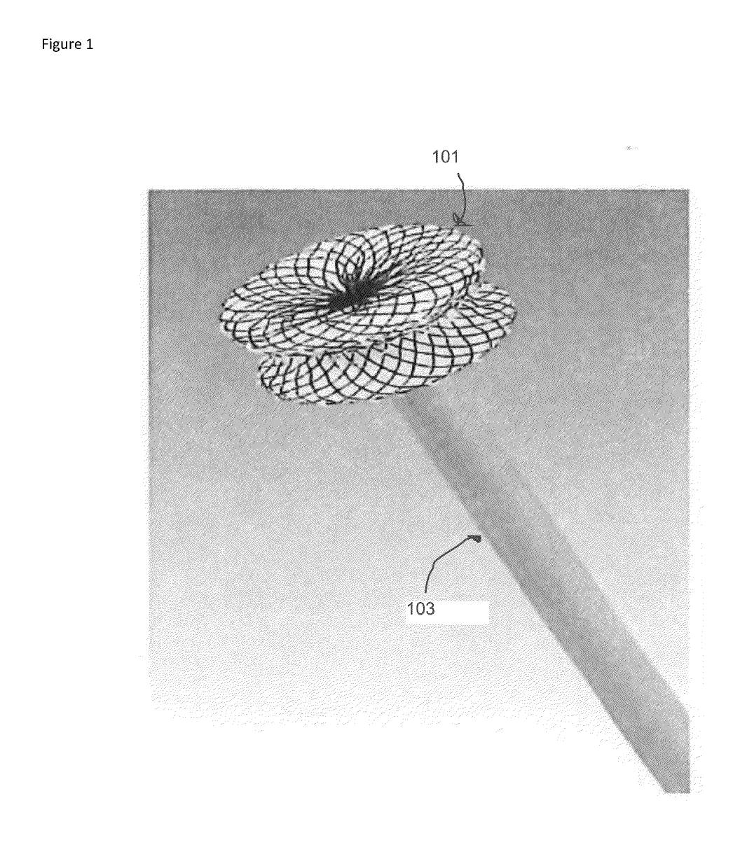

Referring now to FIG. 1, modified hyperboloid 101, having a top and bottom end is shown on catheter based delivery system 103. Those skilled in the art understanding that known applications of the Seldinger technique apply for both fem-fem cut downs and other ingress and egress to the heart. Modified hyperboloid 101 likewise may be a one or two piece 3 assembly and find uniquity, among other things, in being emplaced without application of damaging radial force to the surrounding tissues (which would have a substantial risk of restriction of blood flow around the valve area causing ischemic events or heart attacks). Requirements for the instant invention 101, when emplaced include sealing to the host tissue and not migrating.

As known to artisans, over- and/or undersizing modified hyperboloid 101 is the way the orifice is fitted to the challenged tissue which was the native mitral valve. This facilitates anchoring, and is how the bottom or pressure side of modified hyperboloid 101 interfaces with the chordea in the heart which support the leaflet tissue, anchoring the host/native mitral valve ab initio.

Known biological adhesives and a material effective for tissue attachment/tissue ingrowth are the other pieces of the puzzle which have been denoued according to the instant teachings. Temporary biological adhesive use is wheat is contemplated in combination with clinically proven in-growth materials, such as ePTFE which will promote tissue attachment/tissue ingrowth and long term residence of modified hyperboloid 101, within the patient, so that once healing is complete, a patient may return to normal activities.

Those skilled in the art understand how bottom portion 101 supports tissue ingrowth when emplaced endovascularly, by methods including a femoral-femoral cut-down and catheter.

Pressure side of 101, or bottom portion, integrates with existing/extant chordea, allowing for tissue ingrowth and tissue attachment, making emplacement result in an integral valve assembly over time.

While the method and apparatus have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant.

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A device for replacing the mitral valve, comprising, in combination:
a modified hyperboloid self-expanding stent-like assembly having a top and a bottom end;
operatively linked at least one of a biological adhesive and a porous coating layer;
a catheter-based delivery system; and,
wherein the bottom or pressure side of the assembly is modified to support integration with chordea for anchorage enhancement.

2. The device of claim 1, further comprising ePTFE

3. The device of claim 2, further comprising at least a shape memory alloy.

4. The device of claim 1, wherein the device is two-piece.

5. The device of claim 4, wherein deployment from a catheter and securement in place happens without application of damaging amounts of radial force to surrounding tissue.

6. The device of claim 5, wherein radial force is controlled and migration addressed, enabling long-term emplacement, and enhancement of the quality of life of a patient.

7. The device of claim 1, wherein tissue ingrowth is facilitated by the structure of the device.

8. In a device for mitral valve replacement which facilitates tissue attachment, the improvement which comprises, in combination:
at least a modified hyperoloid stent-like assembly, having a top portion and a bottom portion; wherein the bottom portion supports tissue ingrowth.

9. The device of claim 8, wherein the bottom portion integrates with extant chordea for anchorage attachment.

10. The device of claim 9, wherein said bottom portion is the pressure side of the stent-like assembly.

11. A method of endovascular mitral valve replacement, comprising, in combination:
providing a modified hyperboloid self-expanding stent assembly;
delivering the assembly via a catheter through a femoral-femoral cut-down; and,
emplacing the same,
whereby tissue ingrowth is facilitated.
